(19) United States
(12) Patent Application Publication
(10) Pub. No.: US 2001/0044602 A1
(43) Pub. Date: Nov. 22, 2001

(54) Infusion pump

(76) Inventors: Klaus Angersbach, Morschen (DE); Rolf Heitmeier, Baunatal (DE)

Correspondence Address: DILLER, RAMIK & WIGHT, Suite 101, 7345 McWhorter Place, Annandale, VA, 22003, US

(21) Appl. No.: 09/854,601

(22) Filed: May 15, 2001

(30) Foreign Application Priority Data
May 18, 2000 (DE) 200 08 961.7

(51) Int. Cl. A61M 1/00

(52) U.S. Cl. 604/151; 604/65

(57) ABSTRACT

An infusion pump comprises a mobile self-contained functional basic module (10) with a pump portion and an energy storage means. For more sophisticated programming and performance tasks, the basic module (10) is inserted into an operating module (20) with an input device (27) for more complex inputs. Thus, the infusion pump may be used as a basic module (10) or as a combination of the basic module and the operating module (20).

### BACKGROUND OF THE INVENTION

The present invention refers to an infusion pump for administering liquids to a patient.

Infusion pumps are used for the intravenous administration of drugs, for artificial enteric or parenteric alimentation and for analgetic therapy. Two typical fields of application result from this, namely the stationary use and the mobile use. For both fields of use there are special infusion pumps. For mobile use, mobile infusion pumps have been developed that are rather small and light, however, their performance range is relatively limited with respect to programmability and operating possibilities. Moreover, there are stationary infusion pumps that have a large range of operating possibilities and can be operated by the electrical supply network without time limits.

### SUMMARY OF THE INVENTION

It is the object of the present invention to provide an infusion pump that is adapted both for stationary use and for mobile use, respectively having those properties that are required for the relevant application.

The object is solved, according to the invention, with the features of claim 1. Thus, the infusion pump comprises a basic module forming a self-contained mobile pump, and an operating module with which the basic module can be optionally combined to form a stationary infusion pump with a wider performance range. The operating module is no functional infusion pump by itself. It includes additional elements for an infusion pump such as a computing unit, a storage unit, a data base, a disk drive or a similar reproduction device for data carriers, and in particular an input device that allows for a complex and varied input of infusion parameters. Preferably, the operating module is provided with a display device in the form of a display monitor on which alphanumeric indications are displayed on the one hand, while, on the other hand, graphic representations are shown, such as a time-related profile of the infusion rate, for example. The basic modules are standardized pump modules that have a pump portion with an associated drive and are functional by themselves, though they have limited programming and operating time capacities. Additionally, the operating module is provided that allows for a more universal programming with higher operating comfort.

The present infusion pump can be employed in different stages of a sickness or medical treatment. It allows for a transfer of a chronically ill patient into domestic care, facilitates the mobilization process of a patient in hospital, allows for in-hospital transportation, and is further adapted for maximum intensive medical care. The pump has universal applicability so that no special pumps are required for the different uses. This includes that the patient remains connected to the same hose system, generally one-way hoses, throughout different applications.

The basic module has a minimized user panel and an energy supply of its own; it can further comprise connector elements for connecting additional operating elements. Further, the basic module is provided with the necessary safety means for intravenous application. The minimized user panel means, for example that an alphanumeric display and at most five operating elements are provided. The operating elements can be keys (softkeys) described in their function by means of the display, or they may be mechanical switches or other operating means reacting differently to different actuations. In principle, the setting device comprises a selection device that allows paging up or down a list of selectable operating frames, an acquitting means and an adjusting means for increasing or reducing numerical values.

The data-technical and the mechanical coupling of the basic module to the operating module serves to exchange data and to mechanically fix the basic module. The data exchange can be effected electrically, electromagnetically, e.g. by high frequency, optically, e.g. per infrared interface, by sound, e.g. ultrasonically, or by data carriers. Mechanical connecting elements provide for the mechanical fixation. These can be designed such that the fixation is effected by a guiding that has a locking mechanism and is an integral part of the operating module.

The basic module has an energy storage means of its own that guarantees a self-contained operation during the time of use within the mobility phase. This energy storage means can be replaceable from outside, or it may be permanently integrated. The monitoring of the available capacity and its visualization is effected by the basic module. The energy storage means may be recharged via the operating module or via an additional charging means supplied from the mains. When the basic module is connected to the operating module, the operating module automatically takes over the energy supply.

The basic module has an interface to a remote control. This may be, for example, a patient key switch for delivering boli of analgetics. The remote control may communicate with the basic module either wirelessly or over wires.

### BRIEF DESCRIPTION OF THE DRAWINGS

The following is a detailed description of the invention with reference to the accompanying drawing, wherein:

FIG. 1 illustrates a perspective view of an infusion pump comprising a base module and an operating module;

FIG. 2 is an isolated representation of the basic module,

FIG. 3 illustrates the insertion of the basic module into the operating module under a first orientation of the basic module, and

FIG. 4 illustrates the insertion of the basic module into the operating module under a first orientation of the basic module.

### DETAILED DESCRIPTION OF THE INVENTION

The infusion pump comprises a basic module 10 forming a fully operational mobile infusion pump. The basic module 10 comprises a housing 11 including, for example, a syringe pump or roller pump and a drive device (not illustrated), as well as an energy storage means in the form of an electrical battery. Moreover, the housing includes a control means in the form of a microprocessor. A feed hose 12 leads to the housing 11 from an infusion vessel. A patient hose 13 to which the patient is connected leads away from the housing.

Provided at the front of the housing, there is a user panel 14 having a display 15 with a setting device 16, a selection device 17 and an acknowledgment button 18. The user panel 14 serves to display and to set pump parameters.

The basic module 10 is provided with the safety means required for intravenous application, i.e. measures against unintentional infusion of air, measures against over- or under-dosing, measures against the buildup of too high pressures in the patient hose, and measures against an occlusion of the path between the infusion vessel and the pump module. The pump module 10 is a mobile module that can be fixed to the patient's body, the patient's bed or another mobile object, and it can be carried along without any locally fixed connection. If need be, the basic module can be inserted into a recharging station to recharge the energy storage means.

The basic module 10 may be inserted into an operating module 20 provided for this purpose. The operating module 20 comprises an elongate housing portion 21 with an L-shaped profile forming a bottom wall 22 and a rear housing member 23. The front end of the bottom wall 22 is mounted with a front housing 24 that can be pivoted about an axis 25. In the upright state, the front housing 24 and the L-shaped housing 21 form a U-shaped or groove-shaped structure that fittingly encloses the basic module 10. In doing so, the front housing 24 covers the front of the basic module.

The front housing 24 comprises a display device 26 in the form of a monitor for displaying graphical representations and alphanumerical indications, as well as an input device 27 with an alphanumerical keyboard 28 and operating keys 29. When the basic module is inserted, the operating module is operatively connected with the basic module so that the resulting assembly forms an upgraded functional pump. Compared to the basic module, the operating module has a larger user panel and additional sensor terminals (not illustrated), as well as additional alarm terminals. It includes an extended data base for medicaments and drugs, as well as a data-technical coupling to a central data acquisition system. The operating module is provided with a holder for mounting to a rail on a wall or an infusion stand 29. Moreover, it may have a reproduction device for reading data carriers.

The operating module 20 allows for an adjustable setting of the basic module. It may be used as a programming unit for the basic module. In the operating module, more complex functions are visualized and operated. In general, the operating module is a performance upgrade of the operation, whereas the actual parameter controlled therapy is effected by the basic module and will be continued by the basic module after separation from the operating module.

The operating module can be coupled mechanically with further similar operating modules so that a unit of a plurality of operating modules is obtained.

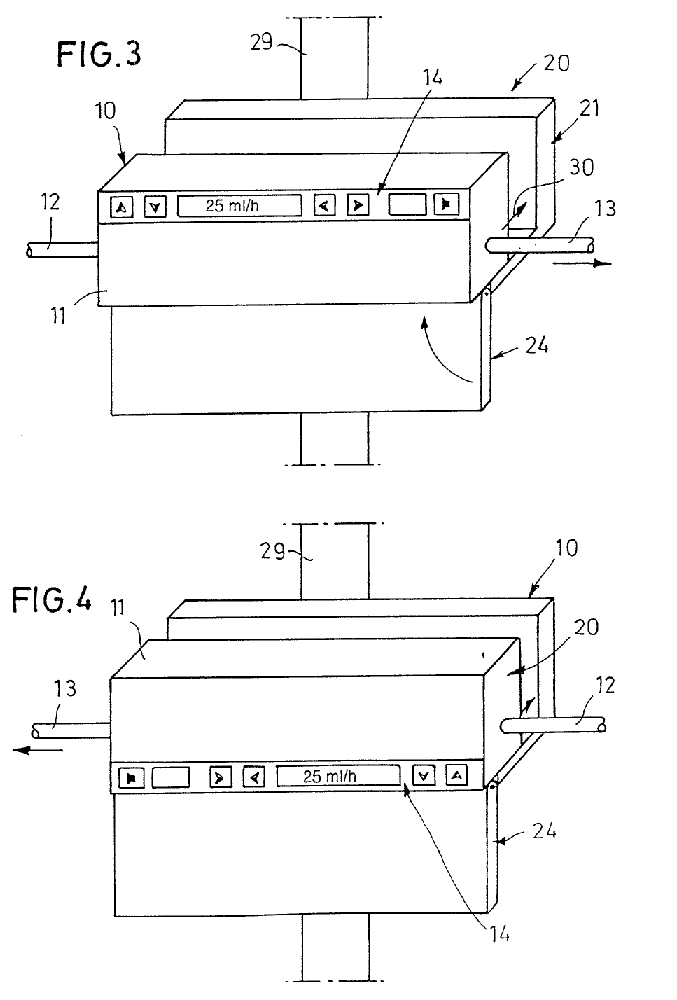

FIG. 3 illustrates the insertion of the pump module into the operating module. Here, the front housing 24 of the operating module is pivoted open. The basic module is pushed into the operating module in the direction of the arrow 10, with the electric connections being made automatically and a mechanical coupling being established. Then, the front housing 24 is pivoted upward so that the state illustrated in FIG. 1 is obtained. In this state, the display and input device of the front housing covers the user panel of the basic module so that the operator cannot erroneously perform settings at the basic module. When the basic module is inserted into the operating module, all settings and display functions are effected only at the operating module.

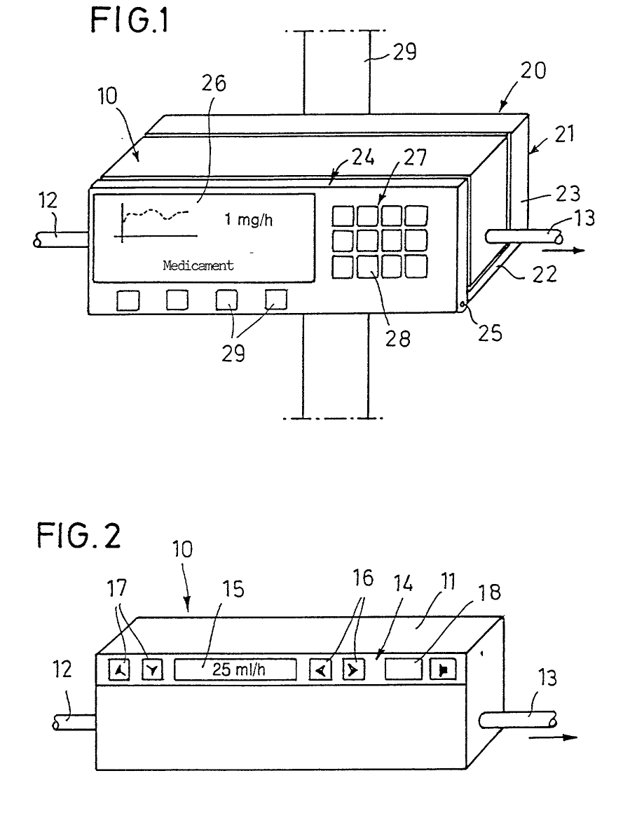

In the basic module 10 of the illustrations of FIGS. 1 to 3, the patient hose 13 extends from the right-hand side of the housing 11. FIG. 4 illustrates the same state as FIG. 3, however, the basic module 20 is structured such that the end from which the patient hose 13 extends is on the left. The user panel 14 arranged at the upper end of the housing in FIG. 3 is disposed at the bottom end of the front in FIG. 4. The housing 11 of FIG. 4 is thus rotated by 180° as compared to the state illustrated in FIG. 3, and the display devices of the user panel 14 are represented upside down so that the display devices can be read under various orientations of the housing 11. The hose guiding can thus be changed selectively corresponding to the position of the patient such that the patient hose 13 extends in different directions.

What is claimed is:

1. An infusion pump with a self-contained functional mobile basic module (10) comprising a pump portion, an energy storage means and a setting device (16), and with an operating module (20) adapted to be coupled to the basic module (10) for transferring energy and data and having an input device (17), the operating module and the basic module together forming an upgraded pump unit.

2. The infusion pump of claim 1, wherein the operating module (20) has sensor terminals.

3. The infusion pump of claim 1 or 2, wherein the operating module (20) has at least one alarm terminal.

4. The infusion pump of claim 1, wherein the operating module (20)includes a data base for medicaments.

5. The infusion pump of claim 1, wherein the operating module (20) has a holder for mounting it to support means (29).

6. The infusion pump of claim 1, wherein the operating module (20) includes a reproduction device for reading data carriers.

7. The infusion pump of claim 1, wherein the basic module (10) has a display device (15) on an elongate housing (11), whose representations may be displayed upside down so that they can readily be read under different orientations of the housing (11).

8. The infusion pump of claim 1, wherein the operating module (20) forms a programming station for the basic module (10).

9. The infusion pump of claim 1, wherein the operating module (20) has a receptacle into which the basic module (10) may be inserted and which covers the setting device (16) of the basic module (10).
